(19) United States
(12) Patent Application Publication
(10) Pub. No.: US 2026/0041864 A1
(43) Pub. Date: Feb. 12, 2026

(54) DYNAMIC CONTROL OF POSITIVE AIRWAY PRESSURE DEVICE

(71) Applicant: 3B Medical, Inc., Dublin, OH (US)

(72) Inventors: David Troxell, Poulsbo, WA (US); Andrew Chapman, Bothell, WA (US); Christopher Chapman, Snohomish, WA (US)

(21) Appl. No.: 19/290,752

(22) Filed: Aug. 5, 2025

Related U.S. Application Data
(60) Provisional application No. 63/681,421, filed on Aug. 9, 2024.

(51) Int. Cl. A61M 16/00; A61M 16/06

(52) U.S. Cl. CPC A61M 16/022; A61M 16/0003; A61M 16/06; A61M 2016/0027; A61M 2016/0033; A61M 2202/0007; A61M 2202/02; A61M 2205/3327; A61M 2205/3334; A61M 2205/50; A61M 2230/005; A61M 2230/40

(57) ABSTRACT

A system may include a respiratory device configured to deliver fluid to a patient. The system may include a controller configured to receive information indicative of a condition of an airway of the patient during an immediately preceding breath of the patient or a current breath of the patient. Further, the controller may be configured to issue one or more commands to adjust a pressure of the fluid delivered to the patient based on the information. A method is also disclosed.

### RELATED APPLICATION(S)

This disclosure claims the benefit of U.S. Provisional Application No. 63/681,421, Filed Aug. 9, 2024, the entirety of which is herein incorporated by reference

### TECHNICAL FIELD

This disclosure relates to dynamic control of a positive airway pressure (PAP) device.

### BACKGROUND

Positive Airway Pressure (PAP) therapy is a treatment for obstructive sleep apnea (OSA), a condition characterized by episodes of airflow reduction (hypopnea) or cessation (apnea) due to upper airway collapse during sleep. PAP therapy involves a PAP device that delivers a stream of air through a patient interface, which is typically provided by a mask worn over the mouth and/or nose. The stream of air delivered by the PAP device helps keep the airway open by preventing hypopnea and apnea events and generally promoting better sleep quality.

### SUMMARY

In some aspects, the techniques described herein relate to a system, including: a respiratory device configured to deliver fluid to a patient; and a controller configured to receive information indicative of a condition of an airway of the patient during an immediately preceding breath of the patient or a current breath of the patient, wherein the controller is configured to issue one or more commands to adjust a pressure of the fluid delivered to the patient based on the information.

In some aspects, the techniques described herein relate to a system, wherein the controller is configured to issue one or more commands such that a pressure of fluid delivered to the patient follows a pressure profile.

In some aspects, the techniques described herein relate to a system, wherein: the pressure profile is set such that the pressure of fluid within the airway of the patient remains at a target pressure, the target pressure is a predetermined amount above a critical pressure adjacency, and the critical pressure adjacency is a pressure at which an airway of the patient experiences a flow limitation but is not collapsed.

In some aspects, the techniques described herein relate to a system, wherein the critical pressure adjacency is a predetermined amount above a critical closing pressure of the airway of the patient.

In some aspects, the techniques described herein relate to a system, wherein the critical pressure adjacency is determined based on the information.

In some aspects, the techniques described herein relate to a system, wherein the critical pressure adjacency is determined during a breath based on one or more of a peak inspiratory flow rate, a peak expiratory flow rate, an inhalation volume, an exhalation volume, an inspiratory time, an expiratory time, a change in a slope of an inspiratory flow rate, a change in a slope of an expiratory flow rate, and a blower speed.

In some aspects, the techniques described herein relate to a system, wherein the controller is configured to adjust the pressure profile during a breath based on the information.

In some aspects, the techniques described herein relate to a system, wherein the critical pressure adjacency is determined based on a difference between peak inspiratory flow rate and peak expiratory flow rate as measured during an immediately preceding breath.

In some aspects, the techniques described herein relate to a system, wherein the controller is configured to adjust the pressure profile for a breath based on the information corresponding to the immediately preceding breath.

In some aspects, the techniques described herein relate to a system, wherein the pressure profile follows a predefined line.

In some aspects, the techniques described herein relate to a system, wherein the controller is configured to adjust the pressure profile based on the information by horizontally or vertically translating the predefined line.

In some aspects, the techniques described herein relate to a system, wherein: during peak inhalation, the pressure profile exhibits a minimum pressure, and during an end section of an exhalation phase, the pressure profile exhibits a maximum pressure.

In some aspects, the techniques described herein relate to a system, wherein, at a beginning of an inhalation phase, the pressure profile exhibits the maximum pressure.

In some aspects, the techniques described herein relate to a system, wherein, at a beginning of an inhalation phase, the pressure profile exhibits a pressure less than the maximum pressure.

In some aspects, the techniques described herein relate to a system, wherein the controller is configured to adjust the pressure profile until a critical pressure adjacency is identified.

In some aspects, the techniques described herein relate to a system, further including: a patient interface configured to conduct a flow of the fluid to the patient through the patient interface.

In some aspects, the techniques described herein relate to a system, further including: a sensor configured to generate signals, wherein the controller is configured to interpret the signals as the information.

In some aspects, the techniques described herein relate to a system, wherein the sensor is one of a pressure sensor and a flow rate sensor.

In some aspects, the techniques described herein relate to a system, wherein: the respiratory device is a positive airway pressure (PAP) device.

In some aspects, the techniques described herein relate to a method, including: adjusting a pressure of fluid delivered to a patient by a respiratory device based on information indicative of a condition of an airway of the patient during an immediately preceding breath of the patient or a current breath of the patient.

### BRIEF DESCRIPTION OF THE DRAWINGS

FIG. 1 illustrates an example positive airway pressure (PAP) therapy system being used by a patient.

FIG. 2 is a flow chart representative of an example method according to this disclosure.

FIG. 3 is a graph of pressure versus time, and illustrates a first example pressure profile.

FIG. 4 is a graph of pressure versus time, and illustrates a second example pressure profile.

### DETAILED DESCRIPTION

This disclosure relates to dynamic control of a positive airway pressure (PAP) device. Among other benefits, this disclosure promotes airway patency dynamically, by responding to signals indicative of airway patency one or more previous breaths of a patient (i.e., the user of the PAP device), and/or from the current breath of the patient. This disclosure does so while minimizing the pressure of the air delivered to the patient. As such, this disclosure delivers air pressure that is proportional to the needs of a patient while also prioritizing patient comfort, making it easier for patients to adhere to their treatment and experience its benefits. These and other benefits will be appreciated from the below description.

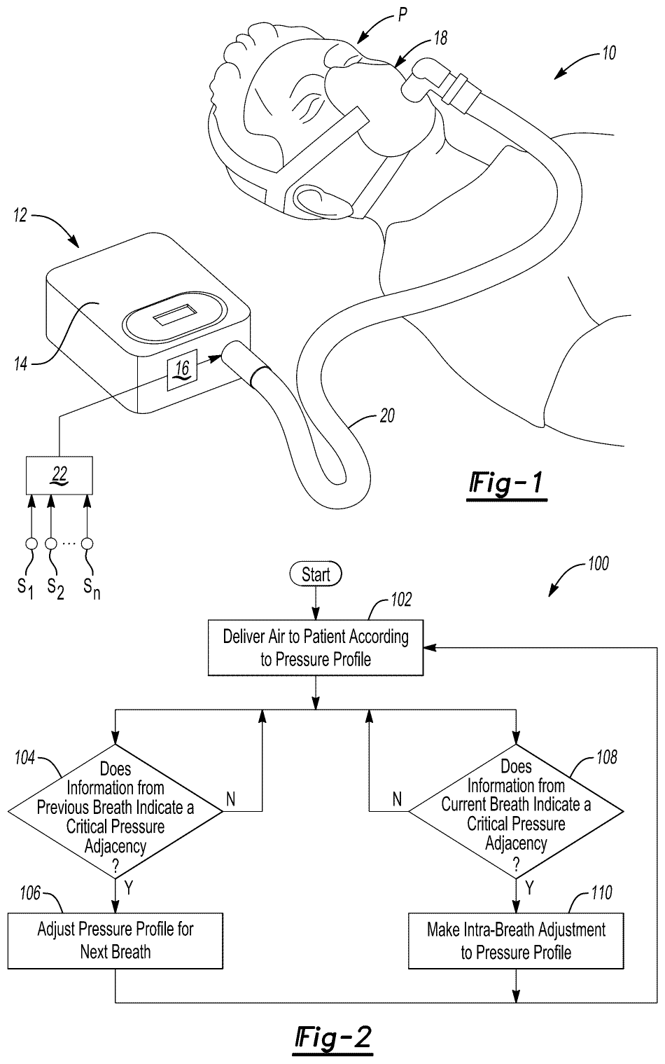

FIG. 1 illustrates an example positive airway pressure (PAP) therapy system 10 (“system 10”) being used by a patient P. The system 10 includes a respiratory device 12, which may be referred to as a PAP device. The term patient as used herein refers to a user of the respiratory device 12. The respiratory device 12 includes a flow generator 14, which includes a blower 16 configured to generate a flow of pressurized fluid, namely air, to be delivered to the patient P. The blower 16 is provided by a motor-driven fan or turbine. The system 10 further includes a patient interface 18 configured to interface with a mouth and/or nose of the patient P. In this example, the patient interface 18 is a full-face mask. This disclosure extends to other types of patient interfaces, including nasal masks and nasal pillows. In this example, headgear is used to secure the patient interface 18. Headgear is not required in all examples. The patient interface 18 is fluidly coupled to the flow generator 14 via a conduit 20. The system 10 may also include a humidifier to add moisture to the air delivered to the patient P.

The system 10 further includes a controller 22. The controller 22 can be incorporated into the respiratory device 12, and in particular may be provided by one or more components mounted within the flow generator 14. The controller 22 includes a memory connected to one or more processors. The memory is configured to store various tables, algorithms, and instructions, which are executable by the processor(s). The processor(s) may be implemented by one or more microprocessors, microcontrollers, application-specific integrated circuits (“ASIC”), digital signal processors (“DSP”), combinations or sub-combinations thereof, or the like. The processor(s) may be integrated into an electrical circuit, such as a conventional circuit board, that supplies power to the processor(s). The processor(s) may include internal memory and/or the memory may be coupled thereto. The present disclosure is not limited by the specific hardware component(s) used to implement the processor(s) and/or the memory.

The memory is a computer readable medium that includes instructions or computer executable components that are executed by the processor(s). The memory may be implemented using transitory and/or non-transitory memory components. The memory may be coupled to the processor(s) by an internal bus.

The memory may include random access memory (“RAM”) and read-only memory (“ROM”). The memory contains instructions and data that control the operation of the processor(s). The memory may also include a basic input/output system (“BIOS”), which contains the basic routines that help transfer information between elements within the respiratory device 12.

Optionally, the memory may include internal and/or external memory devices such as hard disk drives, floppy disk drives, and optical storage devices (e.g., CD-ROM, R/W CD-ROM, DVD, and the like). The respiratory device 12 may also include one or more I/O interfaces (not shown) such as a serial interface (e.g., RS-232, RS-432, and the like), an IEEE-488 interface, a universal serial bus (“USB”) interface, a parallel interface, and the like, for the communication with removable memory devices such as flash memory drives, external floppy disk drives, and the like.

The processor(s) is configured to execute software implementing the processes and control schemes discussed herein, including receiving information from one or more sensors, interpreting information from one or more sensors, and issuing one or more corresponding commands to execute the control schemes discussed herein. Such software may be implemented by the instructions stored in memory.

While a particular embodiment of a respiratory device 12 has been shown in FIG. 1, it should be understood that this disclosure extends to variations of the respiratory device 12, including devices that include lesser or greater functionality relative to the respiratory device 12, and including devices that include fewer or more mechanical components relative to those that are shown in FIG. 1.

The controller 22 is in communication with one or more sensors in this example. As shown in FIG. 1, the controller 22 is in communication with sensors S₁-S_{N}, where “N” is indicative of any number. The sensors S₁-S_{N} may be mounted anywhere within the system 10, including within the flow generator 14, blower 16, patient interface 18, and conduit 20. In an example, the sensors S₁-S_{N} include one or more of pressure sensors configured to measure the air pressure delivered to the patient P, or flow sensors configured to monitor the flow rate of air being delivered. The sensors S₁-S_{N} are configured to generate real time data to the controller 22 for monitoring and adjustments. The signals generated by the sensors S₁-S_{N} are interpretable by the controller 22, and the controller 22 can use the signals generated by the sensors S₁-S_{N} to issue one or more commands to the blower 16 to adjust a pressure and/or flow rate of the air delivered to the patient P, for example. Various example sensors, and the manner in which the controller 22 uses information from these sensors, will be discussed below. The controller 22 is also configured to receive other information, such as signals from a motor of the blower 16, indicating various information corresponding to one or more operating conditions of the blower motor, such as a blower speed in rotations per minute (RPM), as an example.

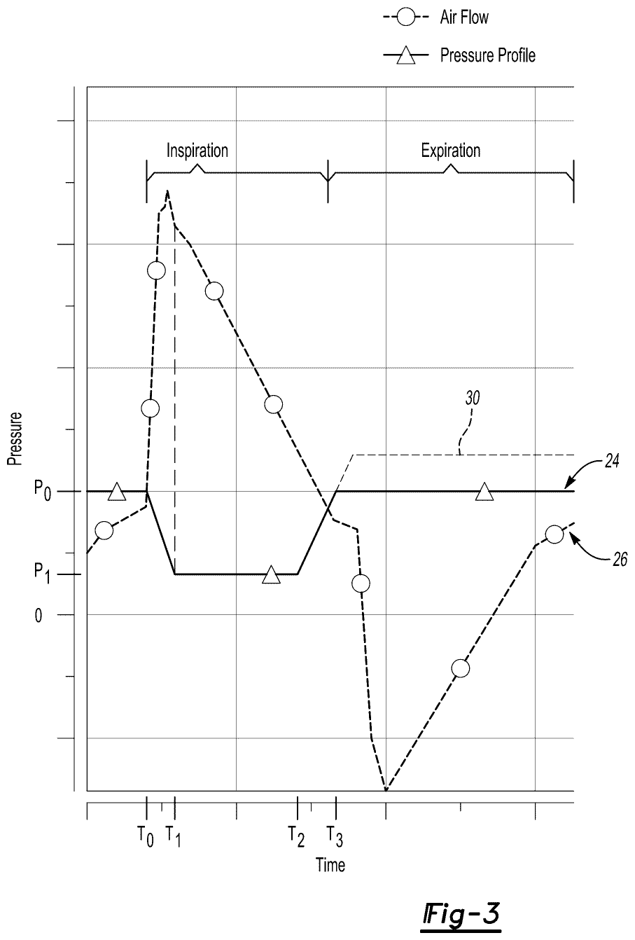

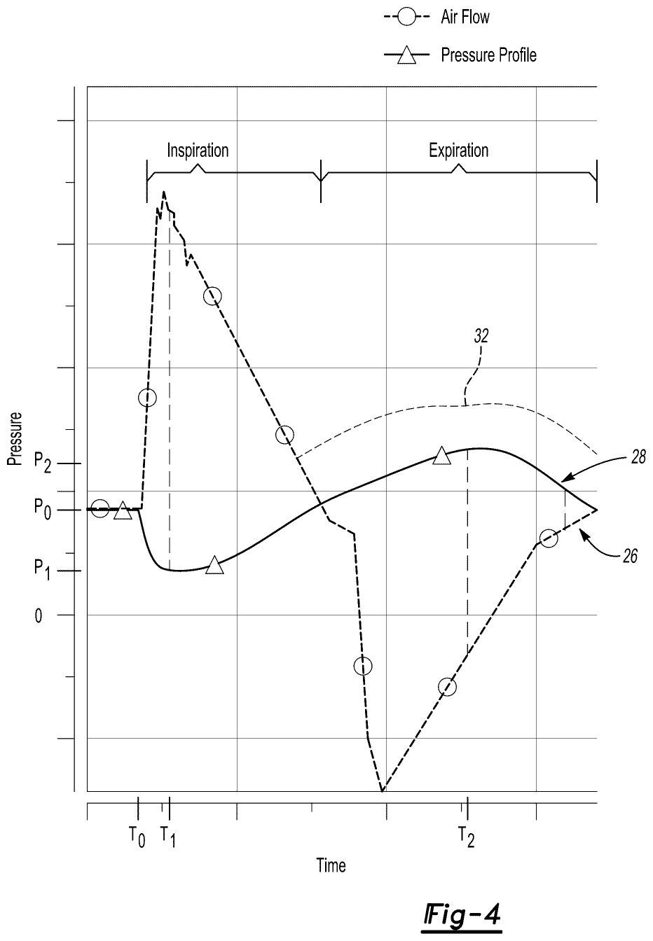

An example method 100 will now be described with reference to the flow chart of FIG. 2 and with reference to the graphical representations of different pressure profiles, as represented in FIGS. 3 and 4. The system 10 may be configured to employ one or more algorithms adapted to execute at least a portion of the steps of the exemplary method 100. For example, the method 100 may be stored as executable instructions in the memory of the controller 22, and the executable instructions may be embodied within any computer readable medium that can be executed by a processor of the controller 22.

In the method 100, during operation of the system 10, the controller 22 is configured to issue one or more commands to deliver a flow of fluid, namely air, to the patient P according to a pressure profile, at 102. The term pressure profile refers to the pressure of the air supplied to the patient, and the pressure profile is variable depending on the stage of a breath cycle, as will be discussed below.

To follow the pressure profile, the controller 22 may issue commands to one or more components of the system 10 to vary the pressure of the fluid delivered to the patient P. In a first example, the controller 22 issues one or more commands to adjust the speed of the blower 16. In another example, the respiratory device 12 may include one or more valves that can be selectively opened and closed to regulate the pressure of the fluid delivered to the patient P.

The controller 22 may include one or more pressure profiles stored in memory. The controller 22 may have a baseline pressure profile, which is initially used and followed. As will be discussed below, the controller 22 is configured to receive information, such as from the sensors S₁-S_{N}, indicative of a condition of an airway of the patient P, and to adjust the pressure profile, if necessary.

The pressure profiles in this disclosure exhibit one or more common characteristics. One example common characteristic is that, during peak inhalation, the pressure profile exhibits a minimum pressure. Another example common characteristic is that, during an end section of an exhalation phase, the pressure profile exhibits a maximum pressure. Without providing the maximum pressure during the end section of the exhalation phase, an airway of the patient P, namely an upper airway of the patient P, may be susceptible to collapse.

Further, the pressure profiles are set such that the pressure of fluid within the airway of the patient P remains at a target pressure (P_{Target}). In this disclosure, target pressure refers to a predetermined amount above a critical pressure adjacency (P_{CritAdj}). The target pressure is a pressure that achieves airway patency via pneumatic splinting. The target pressure may be referred to as an optimal pressure or a controlled pressure. P_{CritAdj} is a pressure at which an airway of the patient P experiences a flow limitation but is not collapsed. A pressure below which the airway of the patient P will experience a collapse is known in the art as a critical closing pressure, or P_{Crit}. P_{Crit} is sometimes referred to as pharyngeal critical closing pressure. P_{CritAdj} is a predefined amount, such as a static amount (e.g., 1 cmH₂0) or a percentage (e.g., 5%), above P_{Crit}. The target pressure is a predefined amount, such as a static amount (e.g., 1 cmH₂0) or a percentage (e.g., 5%), above P_{CritAdj}. By setting the target pressure at a predefined amount above P_{CritAdj}, system 10 avoids P_{Crit} without delivering unduly high pressures to the patient P, which can be uncomfortable and lead to other issues.

The pressure profiles are essentially waveforms representing a pressure at which the pressure of the fluid within the airway of the patient P will be at the target pressure. The controller 22 uses information, such as from the sensors S₁-S_{N}, to determine the pressure of the fluid within airway of the patient P. At least initially, the pressure profiles follow predefined lines. The controller 22 may adjust the pressure profiles depending on the information from the sensors S₁-S_{N} or from other components of the system 10.

A first example pressure profile 24 is shown in FIG. 3 on a graph of pressure versus time. A line 26 representing airflow in and out of the patient P versus time during an example breath cycle is laid over the first example pressure profile 24.

Beginning at the start of inspiration, at time T₀, the controller 22 is configured to command the flow generator 14, such as by commanding the blower 16 to rotate at a particular speed, to deliver a pressure P₀. The pressure P₀ is greater than zero. At time T₁, the line 26 is at a max, indicating a peak inspiratory flow rate. Between times T₀ and T₁, the first example pressure profile 24 gradually reduces, following a constant negatively-sloped line, to pressure P₁. Pressure P₁ is less than P₀ but greater than 0. Pressure is held constant at P₁ between times T₁ and T₂. At time T₂, near an end of the inspiration phase, the first example pressure profile 24 gradually increases, following a constant positively-sloped line back to pressure P₀, at time T₃. Pressure P₀ is then held constant for a majority of the expiration phase, including during an end section of the expiration phase, and is continued until the next breath cycle. In one example, the pressure applied at time T₃ is greater than P₀. In either case, the first example pressure profile 24 is such that the minimum delivered pressure to the patient P is at time T₁, corresponding to a peak inspiration flow rate, and the maximum delivered pressure to the patient P is delivered at an end section of the expiratory phase, at times subsequent to time T₃.

A second example pressure profile 28 is shown in FIG. 4 on a graph of pressure versus time and relative to line 26. Beginning at the start of inspiration, at time T₀, the pressure is at P₀. The pressure P₀ is greater than zero. At time T₁, the line 26 is at a max, indicating a peak inspiratory flow rate. Between times T₀ and T₁, the second example pressure profile 28 gradually reduces, following an exponential decay, to pressure P₁. Pressure P₁ is less than P₀ but greater than 0. Beginning at time T₁, the second example pressure profile 28 gradually increases, following a substantially constant positively-sloped line, to pressure P₂ at time T₂, which is near an end of the expiration phase. Pressure P₂ is greater than pressure P₀. Subsequent to time T₂, the second example pressure profile 28 gradually decreases, following a substantially constant negatively-sloped line back to pressure P₀ at which point the next breath cycle starts. Like the first example pressure profile 24, the second example pressure profile 28 is such that the minimum delivered pressure to the patient P is at time T₁, corresponding to a peak inspiration flow rate, and the maximum delivered pressure to the patient P is delivered at an end section of the expiratory phase, at time T₂.

The controller 22 can follow either the first or second example pressure profiles 24, 28, or another pressure profile. The controller 22 is configured to dynamically adjust the pressure profile that the controller 22 is following based on information relating to a condition of the airway of the patient P. These dynamic adjustments ensure that the system 10 meets the needs of the patient P, which may change throughout a night of sleep, and may even change from breath to breath. Examples of dynamic adjustments will now be described.

In the method 100, at 104, the controller 22 considers whether information from a previous breath, namely an immediately preceding breath, of the patient P indicates that the pressure within the airway of the patient P has reached P_{CritAdj}. P_{CritAdj} may be determined, in one example, by determining a difference between peak inspiratory flow rate and peak expiratory flow rate from the previous breath. If the difference peak inspiratory flow rate and peak expiratory flow rate during the previous breath does not exceed a predefined threshold known to correspond to P_{CritAdj}, then the controller 22 may adjust the pressure profile for the next breath cycle, at 106.

The controller 22 may adjust the pressure profile by horizontally or vertically transforming all or a portion of the pressure profile. For example, if the controller 22 determines that the target pressure needs to be increased, the controller 22 may vertically transform the pressure profile by essentially shifting it vertically upward on a graph of pressure versus time, such as on the graphs of FIGS. 3 and 4.

In the method 100, at 108, the controller 22 considers whether information intra-breath, namely from an ongoing breath of the patient P, indicates that the pressure within the airway of the patient P has reached P_{CritAdj}. P_{CritAdj} may be determined, as non-exhaustive examples, based on one or more of a peak inspiratory flow rate, a peak expiratory flow rate, an inhalation volume, an exhalation volume, an inspiratory time, an expiratory time, a change in the slope of an inspiratory flow rate, a change in the slope of an expiratory flow rate, and/or a blower speed. Other example pieces of information include maximum expiratory flow-volume curve (MEFV) and expiratory time constant (τ). If one or more of these pieces of information crosses a predefined threshold known to correspond to P_{CritAdj}, then the controller 22 will identify a P_{CritAdj} and adjust the pressure profile for the next breath cycle, at 106, or within the current breath, at 110.

As one example, if a peak inspiratory flow rate does not exceed a predetermined threshold equal to 85% of an average peak inspiratory flow rate from a quantity of previous breaths, such as the ten immediately preceding breaths, then the controller 22 will identify a P_{CritAdj} and will adjust the pressure profile for the next breath, or within the current breath. In another example, if an inspiration time does not exceed a predetermined threshold equal to 40% of an average inspiration time from a quantity of previous breaths, then the controller 22 will identify a P_{CritAdj}. In another example, if the inhalation volume is below a predetermined threshold equal to 85% of an average inhalation volume from a quantity of previous breaths, such as the ten immediately preceding breaths, then the controller 22 will identify a P_{CritAdj}. These are merely example thresholds. Further, instead of percentages of averages of previous data, the thresholds could be provided by static values. As one example, if a peak inspiratory flow rate does not exceed a predetermined threshold equal to 0.25 L/sec, then the controller 22 will identify a P_{CritAdj}.

In another non-limiting example, the controller 22 will identify a P_{CritAdj} if the blower speed falls below a lower threshold. In one example, the lower threshold is 18,500 RPM. If the blower speed falls below 18,500 RPM the controller 22 will identify a P_{CritAdj}. In yet another example, if the difference between the maximum blower speed during the inspiratory phase and the minimum blower speed during the expiratory phase falls below a lower threshold, a P_{CritAdj} may be identified.

At 110, the controller 22 is configured to adjust the pressure profile by horizontally or vertically transforming a remaining portion of the pressure profile (i.e., a portion that remains for the ongoing breath). For example, if the controller 22 determines that the target pressure needs to be increased based on some information exceeding a predefined threshold during the inspiratory phase, the controller 22 may vertically transform the pressure profile by essentially shifting the expiratory phase vertically upward on a graph of pressure versus time, such as what is represented by dashed lines 30, 32 in the graphs of FIGS. 3 and 4, respectively.

One aspect of this disclosure relates to determining a viability of a current pressure profile. In this aspect, the pressure delivered to the patient P is gradually reduced until a P_{CritAdj} condition is identified, according to one or more of the above-described techniques. The pressure delivered to the patient P may be reduced iteratively across of series of sequential breath cycles, such as by incrementally vertically transposing a pressure profile downwardly relative to an immediately preceding breath, until a P_{CritAdj} condition is identified. Alternatively, a P_{CritAdj} condition can be identified by gradually reducing the pressure supplied to patient P within a single breath, such as by gradually reducing the pressure supplied to the patient P during an expiration phase. Regardless of the technique, when a P_{CritAdj} is identified, the pressure profile is then set, as discussed above, to deliver a target pressure, which is a predetermined amount above P_{CritAdj}. The method 100 can then be followed, adjusting the pressure profile as necessary.

It should be understood that terms such as “about,” “substantially,” and “generally” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples. In addition, the various figures accompanying this disclosure are not necessarily to scale, and some features may be exaggerated or minimized to show certain details of a particular component or arrangement.

One of ordinary skill in this art would understand that the above-described embodiments are exemplary and non-limiting. That is, modifications of this disclosure would come within the scope of the claims. Accordingly, the following claims should be studied to determine their true scope and content.

1. A system, comprising:
a respiratory device configured to deliver fluid to a patient; and
a controller configured to receive information indicative of a condition of an airway of the patient during an immediately preceding breath of the patient or a current breath of the patient, wherein the controller is configured to issue one or more commands to adjust a pressure of the fluid delivered to the patient based on the information.

2. The system as recited in claim 1, wherein the controller is configured to issue one or more commands such that a pressure of fluid delivered to the patient follows a pressure profile.

3. The system as recited in claim 2, wherein:
the pressure profile is set such that the pressure of fluid within the airway of the patient remains at a target pressure,
the target pressure is a predetermined amount above a critical pressure adjacency, and
the critical pressure adjacency is a pressure at which an airway of the patient experiences a flow limitation but is not collapsed.

4. The system as recited in claim 3, wherein the critical pressure adjacency is a predetermined amount above a critical closing pressure of the airway of the patient.

5. The system as recited in claim 3, wherein the critical pressure adjacency is determined based on the information.

6. The system as recited in claim 5, wherein the critical pressure adjacency is determined during a breath based on one or more of a peak inspiratory flow rate, a peak expiratory flow rate, an inhalation volume, an exhalation volume, an inspiratory time, an expiratory time, a change in a slope of an inspiratory flow rate, a change in a slope of an expiratory flow rate, and a blower speed.

7. The system as recited in claim 6, wherein the controller is configured to adjust the pressure profile during a breath based on the information.

8. The system as recited in claim 5, wherein the critical pressure adjacency is determined based on a difference between peak inspiratory flow rate and peak expiratory flow rate as measured during an immediately preceding breath.

9. The system as recited in claim 8, wherein the controller is configured to adjust the pressure profile for a breath based on the information corresponding to the immediately preceding breath.

10. The system as recited in claim 3, wherein the pressure profile follows a predefined line.

11. The system as recited in claim 10, wherein the controller is configured to adjust the pressure profile based on the information by horizontally or vertically translating the predefined line.

12. The system as recited in claim 10, wherein:
during peak inhalation, the pressure profile exhibits a minimum pressure, and
during an end section of an exhalation phase, the pressure profile exhibits a maximum pressure.

13. The system as recited in claim 12, wherein, at a beginning of an inhalation phase, the pressure profile exhibits the maximum pressure.

14. The system as recited in claim 12, wherein, at a beginning of an inhalation phase, the pressure profile exhibits a pressure less than the maximum pressure.

15. The system as recited in claim 3, wherein the controller is configured to adjust the pressure profile until a critical pressure adjacency is identified.

16. The system as recited in claim 1, further comprising:
a patient interface configured to conduct a flow of the fluid to the patient through the patient interface.

17. The system as recited in claim 1, further comprising:
a sensor configured to generate signals, wherein the controller is configured to interpret the signals as the information.

18. The system as recited in claim 17, wherein the sensor is one of a pressure sensor and a flow rate sensor.

19. The system as recited in claim 1. wherein:
the respiratory device is a positive airway pressure (PAP) device.

20. A method, comprising:
adjusting a pressure of fluid delivered to a patient by a respiratory device based on information indicative of a condition of an airway of the patient during an immediately preceding breath of the patient or a current breath of the patient.
